(19) United States
(12) Patent Application Publication
(10) Pub. No.: US 2022/0007591 A1
(43) Pub. Date: Jan. 13, 2022

(54) NON-DESTRUCTIVE CONDITION ASSESSMENT OF GROWING PLANT MATERIAL

(71) Applicant: Brigham Young University (BYU), Provo, UT (US)

(72) Inventors: Douglas Cook, Fountain Green, UT (US); Brian Mazzeo, Provo, UT (US); Mavrik Thomas, Provo, UT (US)

(21) Appl. No.: 17/302,433

(22) Filed: May 3, 2021

Related U.S. Application Data
(60) Provisional application No. 62/984,633, filed on Mar. 3, 2020.

(51) Int. Cl. A01G 7/04; A01G 7/06; A01G 9/26

(52) U.S. Cl. CPC A01G 7/045; A01G 9/26; A01G 7/06

(57) ABSTRACT

In a general aspect, a method can include measuring at least one electrical property of a growing plant material using a plurality of electrodes to electromagnetically interrogate the growing plant material. The method can further include, based on the measured at least one electrical property, assessing at least one physical condition of the growing plant material.

### CROSS-REFERENCE TO RELATED APPLICATION

This application claims benefit, under 35 U.S.C. § 119, of U.S. Provisional Patent Application No. 62/984,633, filed on Mar. 3, 2020, entitled “Nondestructive Electrical Impedance Condition Assessment of Growing Plant Material”, the disclosure of which is incorporated by reference herein in its entirety.

### TECHNICAL FIELD

This disclosure relates to condition assessment of growing plant material. More specifically, this disclosure relates to non-destructive assessment of growing plant material based on electromagnetic properties of the plant material.

### BACKGROUND

Assessment of health and/or condition of growing plant material, such as hydration levels, damage from pests, etc. can be important for assessing crop health, as well as for determining whether preventive and/or corrective measures should be taken to address undesired plant conditions, such as lack or hydration, presence of pests, etc. However, such assessment is difficult to accomplish without destroying the plant material of interest to make such assessment.

### SUMMARY

In a general aspect, a method can include measuring at least one electrical property of a growing plant material using a plurality of electrodes to electromagnetically interrogate the growing plant material. The method can further include, based on the measured at least one electrical property, assessing at least one physical condition of the growing plant material.

### BRIEF DESCRIPTION OF THE DRAWINGS

FIGS. 1A to 1C are flow diagrams schematically illustrating processes for non-destructive assessment of growing plant material.

FIG. 2 is a diagram that illustrates an experimental setup for measurement of electrical/electromagnetic properties of plant material.

FIGS. 3A, 3B, 4A and 4B are graphs illustrating measured electrical/electromagnetic properties of plant material using, e.g., the experimental setup of FIG. 2, which can be used to assess condition of the plant material.

FIG. 5A to 5C are diagrams illustrating approaches for assessing plant material conditions.

FIGS. 6A, 6B, 7A and 7B are graphs illustrating measured electrical/electromagnetic properties of plant material using, e.g., the experimental setup of FIG. 5A, which can be used to assess condition of the plant material.

FIG. 8 is a graph illustrating capacitance measurements of plant material with varying levels of simulated pest damage.

FIGS. 9A-9B, 10A-10B, 11, and 12A-12B are diagrams illustrating example sensor and/or electrode arrangements that can be used for measuring electrical/electromagnetic properties of plant material.

FIGS. 13A-13B are diagrams illustrating an implementation of an apparatus that can include sensors and/or electrodes for assessing growing plant material condition.

FIG. 14 is a diagram schematically illustrating another apparatus that can include sensors and/or electrodes for assessing growing plant material condition.

FIG. 15 is a diagram schematically illustrating a system for condition assessment of growing plant material.

FIG. 16 is a schematic diagram illustrating a circuit that can be used for performing measurements of electrical/electromagnetic properties of growing plant material.

FIG. 17 is a diagram schematically illustrating a process for performing impedance tomography for condition assessment of growing plant material.

Like reference symbols in the various drawings indicate like and/or similar elements. The drawings are for purposes of illustration and may not necessarily be to scale. Also, in some views, one or more features of an implementation may be obscured or omitted.

### DETAILED DESCRIPTION

This disclosure is directed to non-destructive condition assessment of growing plant material via interrogation of growing plant material using electromagnetic fields. In the approaches described herein, such condition assessment can include determining overall health of a plant, such as moisture content, estimate of internal structure, as well as presence or absence of damage to the plant material, e.g., from pest damage and/or disease. Generally, the electromagnetic properties of a given material can include three parameters: electrical conductivity, σ, dielectric permittivity, ε, and magnetic permeability, μ. For plant material (growing or otherwise), magnetic permeability may be less informative than electrical conductivity (resistance) and/or dielectric permittivity (capacitance), due to the absence of magnetic materials within most plant materials.

These electromagnetic parameters, which are typically unique for different materials, can be dependent on a material's state, such as a hydration level of a material matrix. The values of these parameters for a given material can be, at least in part, due to the intrinsic characteristics of constituent materials that make up a given material. In addition to such intrinsic properties, the geometry of how those constituent materials are arranged can affect resulting electromagnetic field patterns, or electromagnetic interrogation measurements associated with a given material. That is, the same constituent materials, in different arrangements, can have different respective electromagnetic impedance or electromagnetic field characteristics.

Growing plant materials, as their structure changes over time, can also be unique from static materials because growing plant material can have many different structural arrangements over. That is, the material properties of growing plant material change over time as growth of the plant progress. These material properties (e.g., changing properties) can still, however, reflect the health of a growing plant.

This disclosure is directed to approaches, e.g., both apparatus and methods, that can be used to non-destructively assess condition, e.g., health, of growing plant material using electromagnetic interrogation, including electrodes (sensors, etc.) and physical arrangement of electrodes (sensors, etc.) that can be used to interrogate plant material. While example implementations are described with specific reference to maize stalks, it will be appreciated that approaches described herein can be used for non-destructive condition assessment of other plant materials. With respect to maize stalks, non-destructive physical interrogation, e.g., electromagnetic interrogation, can present unique challenges, because maize stalks have different nodes, and numerous leaves emanating from those nodes.

Because non-destructive access can be limited from the top of a maize stalk due the leaves, in the approaches described herein, electrodes or sensors used to perform electromagnetic interrogation can be configured such that they can be placed around, or clamp to a maize stalk without damaging or removing leaves of the stalk. That is, in example implementations, moveable and/or open geometries of an electrode and/or sensor apparatus structure can be used to perform electromagnetic interrogation measurements. For instance a hinged, clamped or fixed arrangements can be used. For purposes of this disclosure, electrodes, sensors, or other elements used for performing electromagnetic interrogation, can be collectively referred to as, e.g., electrode or sensors.

In some implementations, a fixed, U-shaped arrangement of electrodes can be used to gain access to a maize stalk for performing electromagnetic interrogation. That is, in some implementations, positions of electrodes can remain fixed. In some implementations, electrodes included in a electromagnetic interrogation apparatus, can be configured to be moved into position (e.g., hinged, clamped, etc.) for performing electromagnetic interrogation measurements. In some implementations, electrodes for performing electromagnetic interrogation can arranged (e.g., in a measurement apparatus) such that they are aligned parallel to a stalk growth direction, e.g., parallel to a longitudinal axis arranged along a length of a maize stalk.

In implementations, such as those described herein, electrodes can be arranged in planes or can have other geometries, such as half-spheres, where the electrodes can have out-of-plane elements. Such electrodes can be made by using layering or machining to produce geometry and texture that will allow for accurate electromagnetic interrogation measurements of the stalks. In some implementations, electrodes may include rolling elements, such as copper discs, that are able to move up and down a stalk being assessed. Such electrodes can be made to be compliant through the use of various materials, such as conductive foam. In some implementations, electrodes can having restoring forces provided by or more resilient mechanisms or member, e.g., springs or spring-like elements, to provide good contact with the stalk. Electrode elements, such as copper discs or other elements, may have brushes that are spaced around other elements to move along the stalk during movement of an associated electromagnetic interrogation apparatus.

Because of the nodes and the non-uniform surfaces associated with maize stalks, electrodes for electromagnetic interrogation can be used in multiple arrangements to obtain measurement of differing dimensions within a stalk being interrogated. Such approaches can allow for obtaining measurements that can then be used to perform a computed impedance tomography process (e.g., around a stalk, in a plane orthogonal to a stalk growing direction), as well to obtain measurements along the stalk for volume impedance measurements. In some implementations, the electrodes can be regularly spaced, such as in an array, and/or irregularly spaced (with known offsets), such that multiple electromagnetic interrogation measurements can be made with a fixed or variable geometry, with the electrode spacing being considered when making the electromagnetic interrogation measurements.

In some implementations, a part of electromagnetic interrogation apparatus, e.g. a probe or plurality of electrodes can be detached from a main electrode set, so as to form an additional connection to the plant material or to another system such as earth ground, e.g. a ground connection, in order to perform the electromagnetic interrogation measurements. This other electrode system may also have multiple elements, and the distance between it and the other elements may be recorded either automatically or through user interaction, e.g., to be able to measure respective distances between the plurality of electrodes connected to the ground and the interrogation point on the stalk, so as to locate and record the relative positions of the measurements along the stalk.

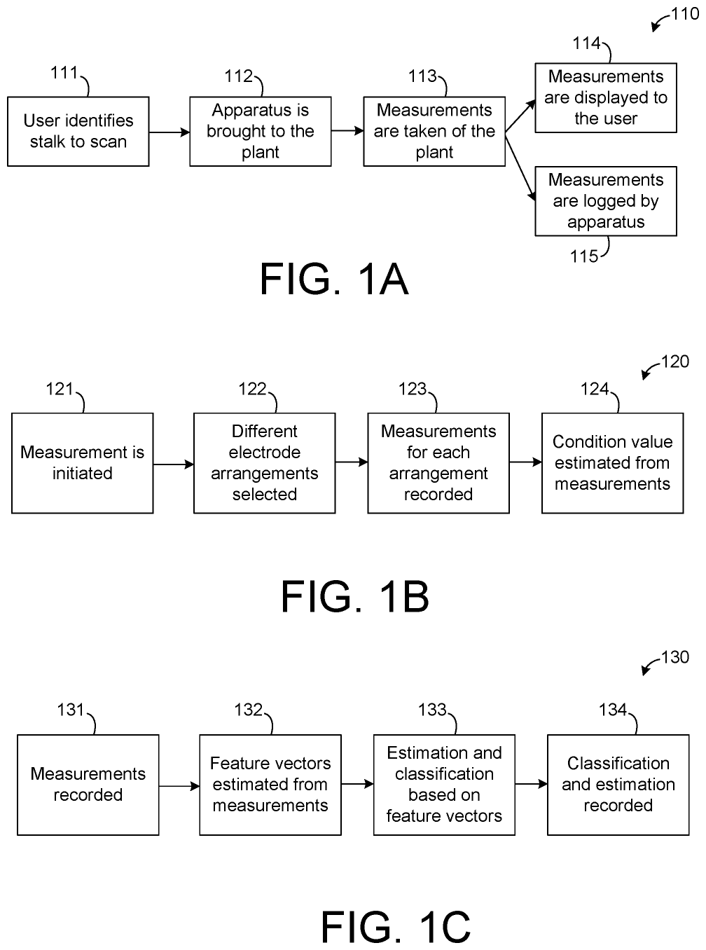

FIGS. 1A to 1C are flow diagrams schematically illustrating processes for non-destructive condition assessment of growing plant material. Specifically, FIGS. 1A to 1C illustrate high level processes, or methods 110, 120 and 130 for performing non-destructive condition assessment of growing plant material. The methods 110, 120 and 130 can be performed using the apparatus and approaches described herein. In some implementations of such methods, electrode element measurements can be produced in real-time, so as to provide feedback to estimate electrode contact with plant material, and/or to determine that measurement locations on the plant may need to be changed or that certain measurements may need to be repeated, such as due to sheathing from leaves causing interference with interrogation measurements. Such real-time feedback can, in some implementations, assist a user in performing the task of plant condition estimation using described techniques.

FIG. 1A illustrates a high-level method 110 for non-destructive condition assessment of growing plant material. The method 110 includes, at block 111, a user identifying, or selecting a plant stalk to scan, such as selecting a growing maize stalk to be assessed. At block 112, an electromagnetic interrogation or measurement apparatus, such as those described herein, can be brought to the selected stalk. At block 113, the method 110 includes taking electromagnetic interrogation measurements of the selected stalk using the measurement apparatus. The measurements of block 113 can be provided to a user (block 114), such as on a display device of an associated computing apparatus, and/or can be logged, e.g., by the measurement apparatus, and/or by an connected computing apparatus (block 115) that is operationally coupled with the measurement apparatus. The presented and/or logged measurements can then be subsequently used to assess condition of the selected stalk, and/or for performing an impedance tomography process to create a map of the stalk structure to assess its condition.

FIG. 1B illustrates another high-level method 120 for non-destructive condition assessment of growing plant material. The method 120 includes, at block 121, initiating an electromagnetic interrogation measurement process of the plant material. At block 122, different electrode arrangements of a plurality of available electrodes can be selected. At block 123, the method 120 includes taking electromagnetic interrogation measurements, such as electrical conductivity and/or dielectric permittivity measurements, for each of the selected electrode arrangements. The measurements of block 123 can then be used to determine and provide a condition value (block 124) that is an estimate of a condition (health) of the plant material. In some implementations, the condition value of block 124 can be determined based on comparison of the measurements of block 123 with respective threshold values, where such threshold values can be based on analysis, e.g., destructive analysis, of like plant material of different conditions, such as healthy, pest damaged, diseased, etc.

FIG. 1C illustrates another high-level method 130 for non-destructive condition assessment of growing plant material. The method 130 includes, at block 131, recording electromagnetic interrogation measurements, such as using multiple arrangements of a plurality of electrodes. At block 132, feature vectors can be determined or estimated from the measurements of block 131. In some implementations, such feature vectors can be used for impedance tomography computation, such as described herein. At block 132, condition estimation and classification of the plant material can be determined based on the feature vectors of block 132. At block 134, the method 130 can include recording the classification and estimation of block 133. The recordation of block 134 can be done within an interrogation apparatus used to perform the measurements of block 132, and/or can be done in an associated computing device operationally connected with the interrogation apparatus (e.g., using a wired or wireless communication channel). In some implementations, the determination or estimation of feature vectors of block 132, the estimation and classification of block 133, and/or the recordation of block 134 can be performed at a time subsequent to acquisition of the measurements at block 131. For instance, the measurement of block 131 can be taken on growing plant material within a crop, and then the determination or estimation of feature vectors of block 132, the estimation and classification of block 133 and/or the recordation of block 134 can be done at a later time, such as in a lab, office, etc.

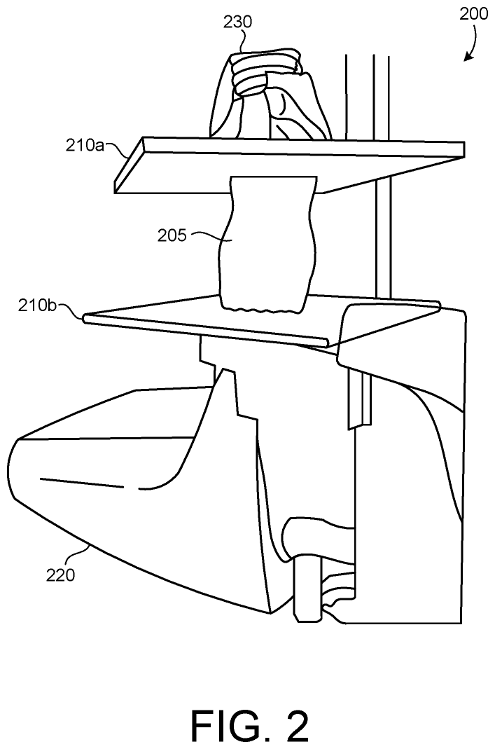

FIG. 2 is a diagram that illustrates an experimental setup 200 for measurement of electrical/electromagnetic properties of plant material. In the example implementation of FIG. 2, a section of a plant material stalk 205, such as a maize stalk, is disposed between a first conductive plate 210a and a second conductive plate 210b, which can be respective copper plates, or guarded electrodes, depending on the particular implementation. In the experimental setup 200, the plant material stalk 205 can be held in place between the first conductive plate 210a and the second conductive plate 210b using a vise 220 and a clamp 230. Electrical connections can be made from the first conductive plate 210a and the second conductive plate 210b to a measurement device, such as parametric analyzer or other device, to perform electromagnetic interrogation of the plant material stalk 205.

While the experimental setup 200 is used to perform destructive analysis of the plant material stalk 205, longitudinal (bulk) electromagnetic interrogation measurements obtained using the experimental setup 200 (e.g., along a longitudinal axis of the plant material stalk 205) can be used to assess electromagnetic parameters of plant material in known states, such as with various levels of hydration. Such assessments can then inform non-destructive analysis using electromagnetic interrogation of like plant material to estimate a condition (health) of a growing plant, such as to determine threshold electromagnetic interrogation values associated with specific plant conditions.

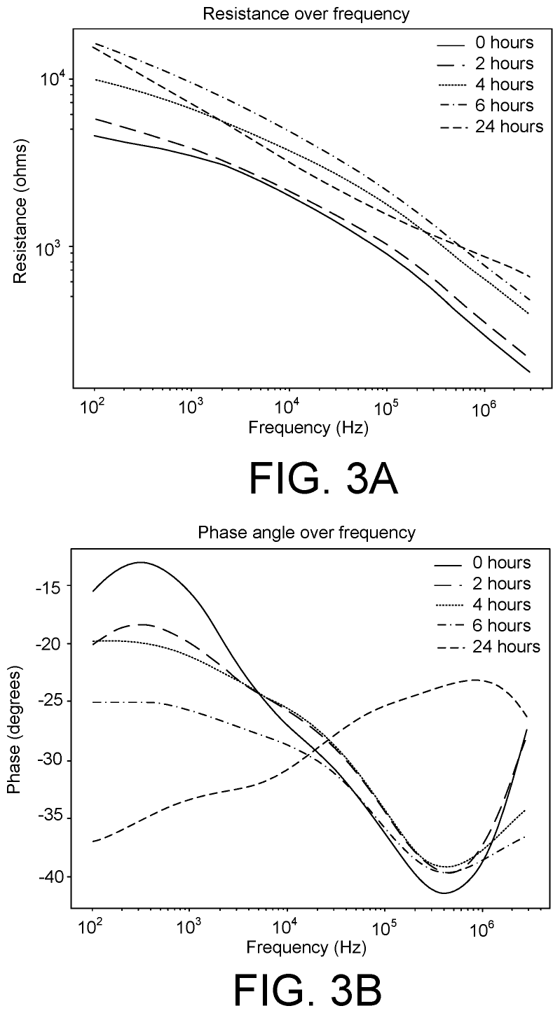

FIGS. 3A, 3B, 4A and 4B are graphs illustrating measured electrical/electromagnetic properties of plant material using, e.g., the experimental setup of FIG. 2. Specifically, FIGS. 3A and 3B illustrate data corresponding with electromagnetic interrogation of a node of a maize stalk taken over time, which, in turn, demonstrates the effects of drying (hydration level) on electromagnetic impedance characteristics of maize stalk node. For instance, FIG. 3A illustrates the relationship of hydration level with electrical resistance of maize stalk node at 0 hours of drying time (e.g., 0 hours after obtaining the maize stalk node from a growing plant), 2 hours of drying time, 4 hours of drying time, 6 hours of drying time, and 24 hours of drying time. As can be seen from FIG. 3A, resistance increases with drying time (e.g., with lower hydration levels). FIG. 3B illustrates the relationship of hydration level with phase angle of the maize stalk node at 0 hours of drying time (e.g., 0 hours after obtaining the maize stalk node from a growing plant), 2 hours of drying time, 4 hours of drying time, 6 hours of drying time, and 24 hours of drying time. As can be seen from FIG. 3B, measured phase and of the maize stalk node changes with drying time (e.g., is dependent on the hydration level within the maize stalk node).

Similar to FIG. 3A, FIG. 4A illustrates the relationship of hydration level with electrical resistance of a maize stalk internode region at 0 hours of drying time (e.g., 0 hours after obtaining the maize stalk internode region from a growing plant), 2 hours of drying time, 4 hours of drying time, 6 hours of drying time, and 24 hours of drying time. As can be seen from FIG. 4A, as in FIG. 3A, resistance increases with drying time (e.g., with lower hydration levels). FIG. 4B illustrates the relationship of hydration level with phase angle of the maize stalk internode region at 0 hours of drying time (e.g., 0 hours after obtaining the maize stalk internode region from a growing plant), 2 hours of drying time, 4 hours of drying time, 6 hours of drying time, and 24 hours of drying time. As can be seen from FIG. 3B, phase angle of the maize stalk internode region changes with drying time (e.g., is dependent on the hydration level within the maize stalk node).

As shown by, at least, FIGS. 3A, 3B, 4A, 4B, a measurement frequency associated with electromagnetic interrogation of plant material may allow for distinguishing between different plant material conditions, such as hydrating levels. In some implementations, multiple frequencies can be tested sequentially (as in a sweep), in parallel (as in simultaneously), or one or more discreet frequencies can be selected at which to perform electromagnetic interrogation to be able to distinguish different plant characteristics.

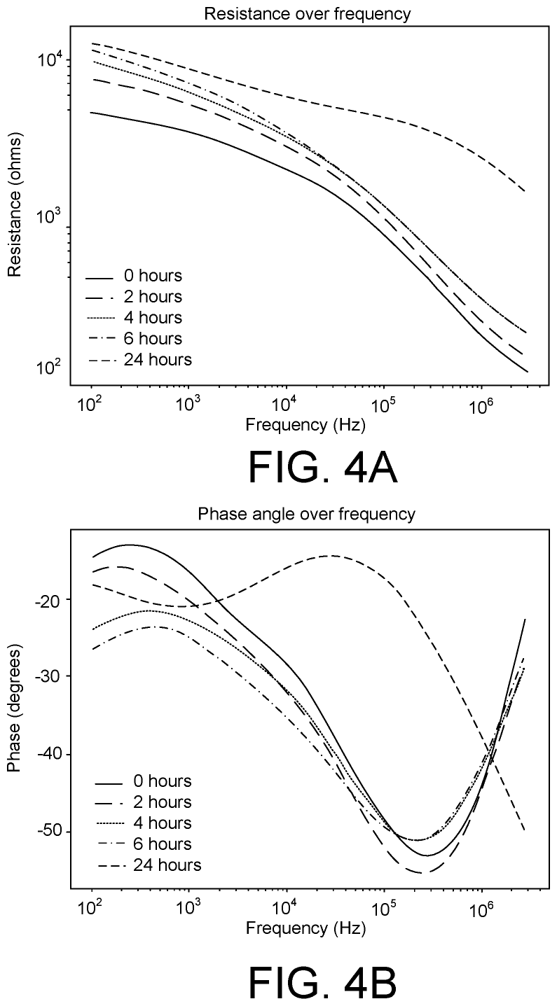

FIG. 5A to 5C are diagrams illustrating approaches for assessing plant material conditions. For instance, FIG. 5A illustrates an experimental for measurement of electrical/electromagnetic properties of plant material. In the example implementation of FIG. 5A, a section of a plant material stalk 505, such as a maize stalk, is disposed between a first conductive plate 510a and a second conductive plate 510b, which can be respective copper plates, or guarded electrodes, depending on the particular implementation. In the experimental setup of FIG. 5A, the plant material stalk 505 can be held in place between the first conductive plate 510a and the second conductive plate 510b using a vise 520 and a clamp 530. Electrical connections can be made from the first conductive plate 510a and the second conductive plate 510b to a measurement device, such as parametric analyzer or other device, to perform electromagnetic interrogation of the plant material stalk 505.

While the experimental setup of FIG. 5A is illustrated as being used to perform destructive analysis of the plant material stalk 505, as with measurements made using the experimental setup 200, transverse electromagnetic interrogation measurements obtained using the experimental setup of FIG. 5A (e.g., orthogonal to a longitudinal axis of the plant material stalk 505) can be used to assess electromagnetic parameters of plant material in know states, such as with various levels of hydration. Such assessments can then inform non-destructive analysis using electromagnetic interrogation of like plant material to estimate a condition (health) of a growing plant, such as to determine threshold electromagnetic interrogation values associated with specific plant conditions. Such non-destructive analysis could be performed using a similar arrangement as shown in FIG. 5A.

FIG. 5B illustrates the plant material stalk 505 with a hollow portion 507 formed in the plant material stalk 505 to simulate pest damage, such as damage that may occur in a maize stalk as a result of a European corn borer (ECB) infestation. FIG. 5C illustrates an approach for creating the hollow portion 507 by inserting rigid wire 540 into the plant material stalk 505 to simulate damage that can be caused in a maize stalk by an ECB. Using the approaches described herein to detect the presence of an ECB infestation can be done using non-destructive approaches can reduce crop loss to both such an infestation as well as destructive analysis of growing crops. Impedance spectroscopy tests performed on both a maize stalk and a celery stalk, before and after forming a hollow portion formed in their respective centers, e.g., such as illustrated in FIGS. 5B and 5C, demonstrated that electromagnetic interrogations measurements are useful in detecting changes in geometry within the plant stalks (e.g., detecting pest damage or disease).

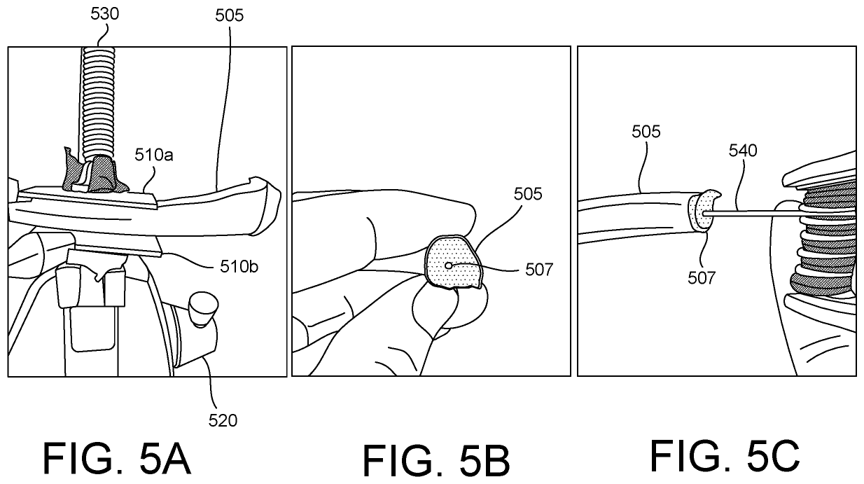

For instance, FIG. 6A illustrates a comparison of capacitance over frequency for a maize stalk prior to hollowing out a center (filled) and after hollowing out the center (hollow), where the measurements were taken using the experimental setup of FIG. 5A. As can be seen in FIG. 6A, a difference in capacitance is observed between the hollow maize stalk and the filled maize stalk. FIG. 6B, illustrates a comparison of resistance over frequency for the maize stalk of FIG. 6A both prior to hollowing out a center (filled) and after hollowing out the center (hollow), where the measurements were taken using the experimental setup of FIG. 5A. As can be seen in FIG. 6B, a difference in resistance is observed between the hollow maize stalk and the filled maize stalk.

FIG. 7A illustrates a comparison of capacitance over frequency for a celery stalk prior to hollowing out a center (filled) and after hollowing out the center (hollow), where the measurements were taken using the experimental setup of FIG. 5A. As can be seen in FIG. 7A, as with the maize stalk of FIGS. 6A and 6B, a difference in capacitance is observed between the hollow maize stalk and the filled maize stalk. FIG. 7B, illustrates a comparison of resistance over frequency for the celery stalk of FIG. 7A both prior to hollowing out a center (filled) and after hollowing out the center (hollow), where the measurements were taken using the experimental setup of FIG. 5A. As can be seen in FIG. 7B, as with the maize stalk of FIGS. 6A and 6B, a difference in resistance is observed between the hollow celery stalk and the filled celery stalk.

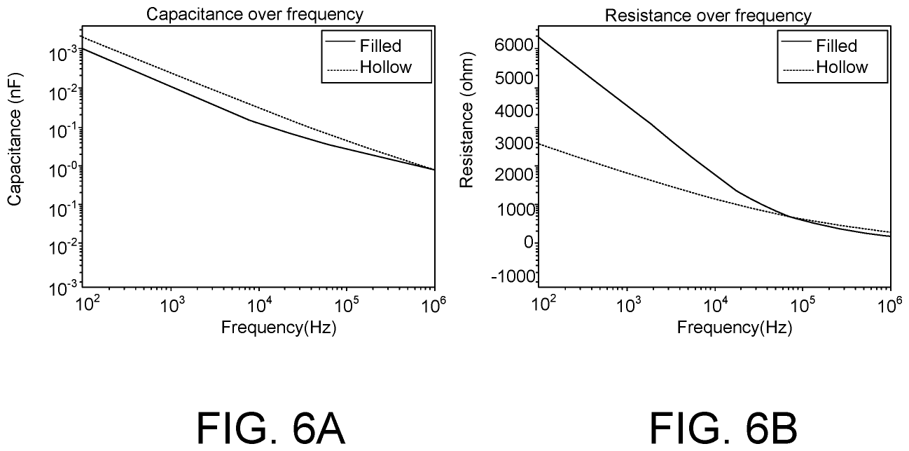

FIG. 8 is a graph illustrating capacitance measurements of plant material with varying levels of simulated pest damage. Specifically, the data in FIG. 8 represents capacitance data taken at 100 Hz on two maize stalk samples, Stem 1 and Stem 2. In the graph of FIG. 8, individual data points (respectively, filled and open stars) are shown for each stalk sample. Further, the lines in FIG. 8 represent respective least squares fits for the respective maize stalk data measurements for Stem 1 and Stem 2. The empirical measurements for Stem 1 and Stem 2 were taken starting with no simulated damage, and then at increasingly larger hole sizes, where the holes were formed longitudinally in the center of the respective stalks, such as using the approach shown in FIG. 5C.

The damage to the stalks of FIG. 8 simulates damage caused by an ECB infestation. As shown by the data of FIG. 8, for both samples, measured capacitance changes in correspondence with hole size. As also shown by FIG. 8, change in capacitance for a given amount of damage (e.g., hole size) can vary from sample to sample, which can be due to a number of factors, such a ratio of hole size to stalk width, a level of hydration of a stalk, etc. However, as shown for Stem 1 and Stem 2 in FIG. 8, capacitance measured for a given stalk is a function of an extent of damage, or hole size present in the stalk.

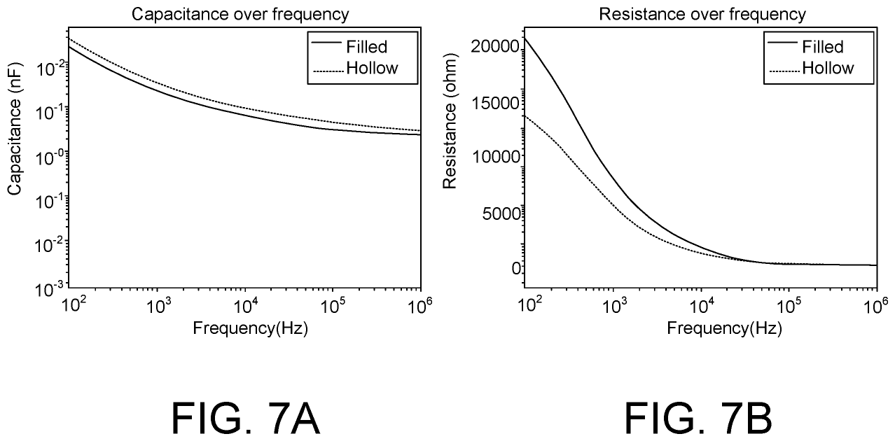

FIGS. 9A-9B, 10A-10B, 11, and 12A-12B are diagrams illustrating example sensor and/or electrode arrangements that can be used for measuring electrical/electromagnetic properties of growing plant material. As discussed above, electromagnetic interrogation tests demonstrate that measures of maize stalk health including hydration level and bore hole size can influence the electromagnetic impedance of the stalks in ways that are associated maize stalk health, pest damage and disease states. Using more than one pair of electrodes, or a plurality of electrodes in synchronized arrangements and patterns to perform electromagnetic interrogation can allow for different interrogation measure to be performed, which can improve the accuracy of estimating a condition of growing plant material, such as allowing for impedance tomography mapping.

FIGS. 9A and 9B illustrate different electromagnetic interrogation configurations that can be performed using four electrodes (e.g., two electrode pairs). As shown in FIGS. 9A and 9B, electromagnetic interrogation of a plant stalk 905 can be performed using a first electrode 910a, a second electrode 910b, a third electrode 910c and a fourth electrode 910d. The arrangement shown in FIGS. 9A and 9B is given by way of example, and for purposes of illustration. In some implementations, a different number of electrodes can be used (more or fewer) and/or different arrangements (e.g. spacing) of electrodes can be used.

In FIGS. 9A and 9B, field lines 915 are shown to represent electromagnetic interrogation fields between respective electrode pairs. For instance, as shown in FIG. 9A, a first electromagnetic interrogation measurement can be performed between the first electrode 910a and the third electrode 910c, while a second electromagnetic interrogation measurement can be performed between the second electrode 910b and the fourth electrode 910d. In the example of FIG. 9B, a first electromagnetic interrogation measurement can be performed between the first electrode 910a and the second electrode 910b, while a second electromagnetic interrogation measurement can be performed between the third electrode 910c and the fourth electrode 910d.

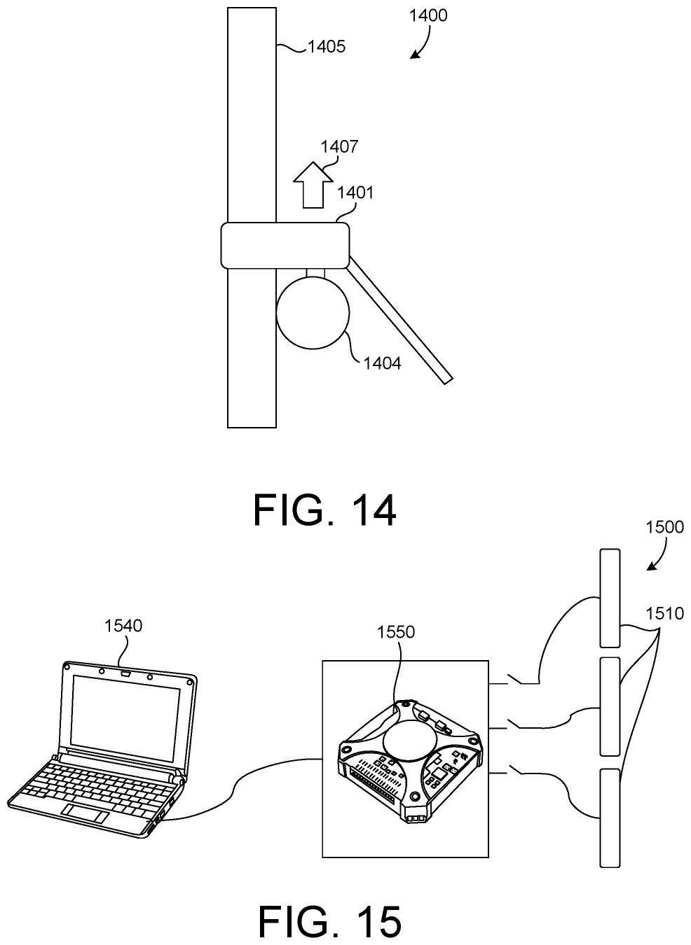

In some implementations, other combinations (e.g., pairs, or groups) of the electrodes of the FIGS. 9A and 9B (or other groups of electrodes, such as the examples described herein) can be used to perform an electromagnetic interrogation measurement. For instance, in an example implementation, an electromagnetic interrogation measurement can be made between the first electrode 910a and the fourth electrode 910d, while the second electrode 910b and the third electrode 910c are floating, or used as guards (e.g., driven to a sense voltage of an associated measurement circuit, such as the circuit of FIG. 16. In some implementations, other arrangements of electrodes used to perform electromagnetic interrogation measurements can be used.

FIG. 10A illustrates an electromagnetic interrogation apparatus 1000 that includes electrodes 1010 arranged similarly to the electrodes of FIGS. 9A and 9B, with six electrodes (rather than two) on each side of a plant stalk 1005 that can be used in various combinations to electromagnetically interrogate the plant stalk 1005. For instance, the electromagnetic interrogation apparatus 1000 can be used to perform two-sided electromagnetic interrogation measurements, e.g., using one of the electrodes 1010 located above the plant stalk 1005 and one of the electrodes 1010 located below the plant stalk 1005. The right side of FIG. 10A illustrates an end view of the electromagnetic interrogation apparatus 1000, which shows a U-shaped member that can placed around the plant stalk 1005 to position the electrodes 1010 in position to perform electromagnetic interrogation measurements on the plant stalk 1005. As in FIGS. 9A and 9B, field lines 1015 are shown to represent electromagnetic interrogation fields between respective electrode pairs.

FIG. 10B illustrates an electromagnetic interrogation apparatus 1050 that includes electrodes 1010 arranged similarly to the upper electrodes 1010 of FIG. 10A. The electrodes 1010 of the electromagnetic interrogation apparatus 1050 can be used to perform single-sided electromagnetic interrogation measurements, such as along the respective paths illustrated by the arrows 1012 in FIG. 10B. In some implementation, the arrangement in FIG. 10B can be implemented by the electromagnetic interrogation apparatus 1000, e.g., by the electrodes 1010 located above the plant stalk 1005 in FIG. 10A (or by the electrodes 1010 located below the plant stalk 1005). The right side of FIG. 10B illustrates an end view of the electromagnetic interrogation apparatus 1050, which shows a half U-shaped member that can placed adjacent to the plant stalk 1005 to position the electrodes 1010 in position to perform electromagnetic interrogation measurements on the plant stalk 1005. As in FIGS. 9A, 9B and 10A, field lines 1015 are shown to represent electromagnetic interrogation fields between respective electrode pairs.

As noted above, various combinations of the electrodes 1010 of FIGS. 10A and 10B can be used to perform electromagnetic interrogation measurements on the plant stalk. For instance, in FIG. 10A, there are 132 possible two electrode combinations, while in FIG. 10B there are 30 possible two electrode combinations. Of course, arrangements where more than two electrodes are used to perform an electromagnetic interrogation measurement are possible. The arrangements of electrodes in FIGS. 10A and 10B allows for performing electromagnetic interrogation measurements along different planes and/or vectors with the plant stalk 1005 of these examples.

In some implementations, the electrodes of electromagnetic interrogation apparatuses described herein can use capacitive sensing to non-destructively assess condition (health) of growing plant material. For instance, capacitive sensing can be used to detect capacitive differences within a stalk, which can be used to identify changes in hydration and/or voids (damage) within a stalk. Such capacitive sensing can also be used to identify node and inter-node regions of a maize stalk. In some implementations, such as the examples of FIGS. 9A, 9B, 10A and 10B, electromagnetic interrogation measurements can be performed without corresponding electrodes being in direct contact with plant material being assessed. In such implementations, using an AC signal to make electromagnetic interrogation measurements will result in any air gaps acting as capacitors in series with respective electrodes.

In some implementations, electrodes used for making electromagnetic interrogation measurements can be in direct contact with growing plant material that is being assessed. Such electrodes can also have insulated material covering them to provide abrasion resistance during operation of an associated electromagnetic interrogation device or apparatus.

FIG. 11 is a diagram of an electromagnetic interrogation apparatus 1100 that includes electrodes 1110 configured to being in direct contact with a maize stalk 1105 (or other plant material) being assessed. In some implementations, a coupling fluid can be used to ensure good electrical contact between the electrodes 1110 and the maize stalk 1105. In some implementations, a coupling fluid can be excluded. As with FIG. 10A, the right side of FIG. 11 illustrates an end view of the electromagnetic interrogation apparatus 1100, which shows a U-shaped member that can placed around the plant stalk 1105 to position the electrodes 1110 in position to be place in contact with the maize stalk 1105 (e.g., using a hinge or clamp mechanism) to perform electromagnetic interrogation measurements on the plant stalk 1105.

In some implementations, such as described approaches, electrodes can include different conductive elements, such as conductive brushes or other compliant, electrically conducting material that is suitable for use as an electrode for performing electromagnetic interrogation measurements. FIGS. 12A and 12B are diagrams illustrating an experimental setup where conductive brushes 1210 (e.g., copper brushes) are used for performing electromagnetic interrogation measurements on a plant stalk 1205. In such implementations, brush durability and electrical contact quality with plant material being assessed are important factors for obtaining reliable measurements.

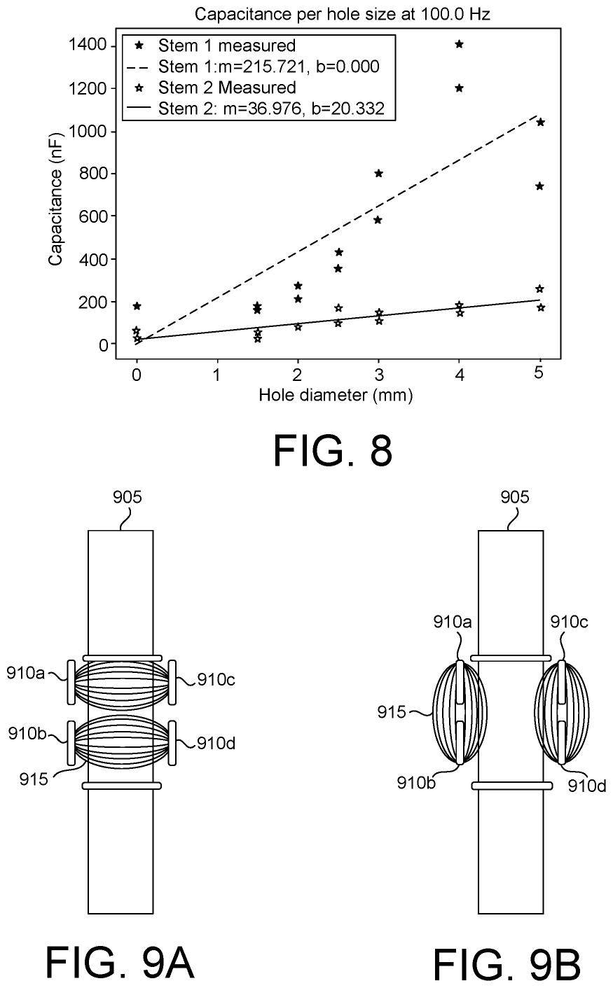

FIGS. 13A and 13B are diagrams illustrating an implementation of an electromagnetic interrogation apparatus 1300 that can include sensors and/or electrodes for assessing growing plant material condition. While not specifically shown in FIGS. 13A and 13B, the electromagnetic interrogation apparatus 1300 can include electrode arrangements, such as those described herein. The electromagnetic interrogation apparatus 1300 can also include other wiring and circuitry, such as multiplexers, transimpedance amplifiers, measurement circuits, etc., which can be used to perform electromagnetic interrogation measurements, impedance tomography and/or assess heath of growing plant material.

In the example of FIG. 13A and 13B, sensors (and other circuitry and/or wiring) can be included on, or within a U-shaped member 1301. The U-shaped member 1301 can be coupled with a handle 1302 via a hinge 1303. The handle 1302 can be used to locate the electromagnetic interrogation apparatus 1300 in proximity of a growing plant to be assessed, and the hinge 1303 can be used to position the U-shaped member 1301 to located included sensors in proximity with, or in contact with growing plant material on which electromagnetic interrogation measurements are to be made. That is, the hinge 1303 can be configured to allow for changing a position of a sensor array included in the u-shaped member 1301 relative to an interior space. The hinge 1303 can also be configured to allow for movement in a single degree of freedom, such as linear travel or in rotational movement, or allow a combination of more than one degree of freedom of movement.

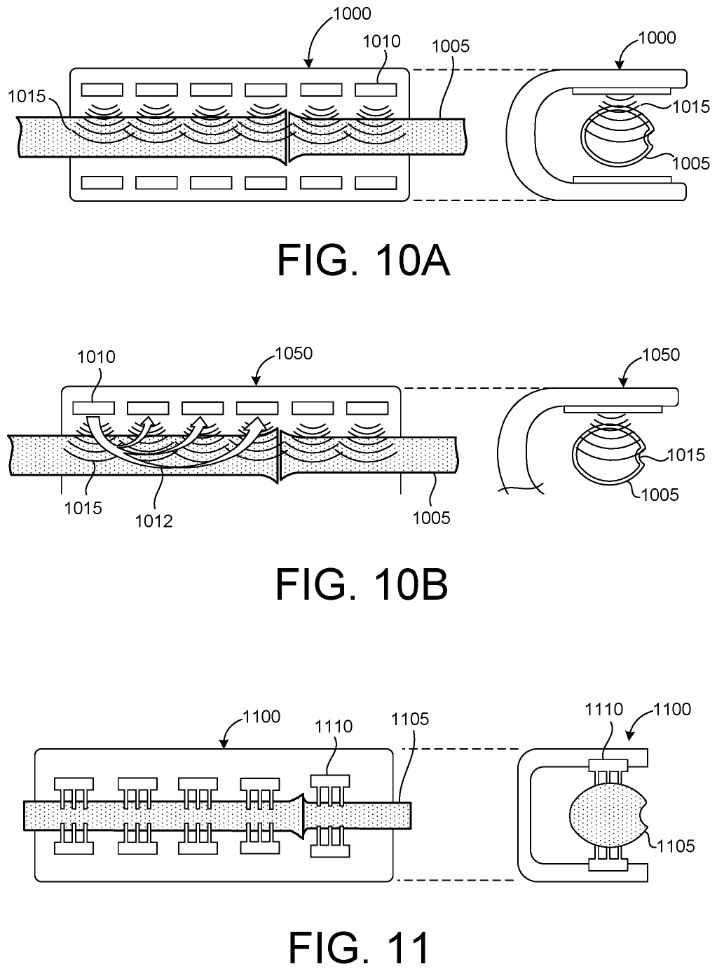

FIG. 14 is a diagram schematically illustrating another apparatus 1400 that can include sensors and/or electrodes for assessing growing plant material condition. In this example, the apparatus 1400 can be used to non-destructively assess condition of a growing plant stalk 1405 (e.g., a maize stalk). As shown in FIG. 14, the apparatus 1400 can include an electrode array 1401 and a distance encoder 1404. In this example, the distance encoder 1404 can be configured to perform location estimation (e.g., estimation of a position on the growing plant stalk 1405) and recording of movement up and down the stalk, such as indicated by the directional arrow 1407. The distance encoder 1404 can be implemented using a distance wheel, using a computed optical metrology approach, or using a LiDAR, ultrasonic, pressure, or other sensing unit. For example, in some implementations, a LiDAR, ultrasonic, or other sensor can be incorporated in the electrode array 1401 in order to measure a distance to the ground or to other reference objects. Such distance information can be used in combination with electromagnetic interrogation measurements to allow for assessing any variations in condition (health) of the growing plant stalk 1405 along its length.

In some implementations, electrical measurements can be used to determine a distance along the growing plant stalk 1405, or from the ground. In some implementations, the electrodes themselves, or an additional rotary encoder may be used to measure the distance traveled along the stalk. This distance determination may also be performed using visual odometry, using computer vision, and/or using a positioning system such as a real time kinematics—global positioning system (RTK-GPS) in order to establish a position along a plant stalk. Position may also be calculated relative to the ground, and/or relative to other objects of interest, such as field markers, fences, buildings and/or other markers that establish position in a field and/or a position on the plants themselves.

In some implementations, the electrode array 1401 can also include elements, such as LEDs or cameras, associated with the electromagnetic interrogation measurements, which are configured to estimate the position of the growing plant stalk 1405 within, or near the electrode arrangement. This estimation may also be accomplished using other physical elements, such as digital calipers, linear encoders, and/or rotary encoders to estimate a position and size of the growing plant stalk 1405 within the electrode array 1401. In some implementations, the apparatus 1400 (or other interrogation apparatuses) can include robotic elements that are configured to move the apparatus 1400 along the growing plant stalk 1405, and/or between stalks.

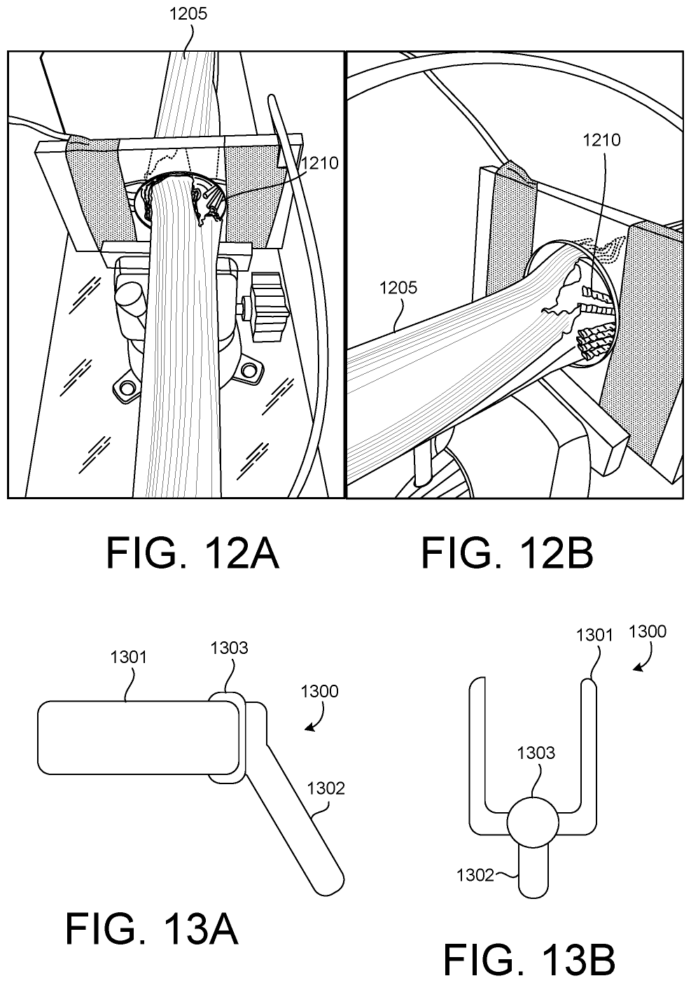

FIG. 15 is a block diagram schematically illustrating a system 1500 for condition (health) assessment of growing plant material. The system 1500 of FIG. 15 includes electrodes 1510, which can be electrodes for performing electromagnetic interrogation measurements, such as those described herein.

The system 1500 can also include circuitry 1550 that is operationally coupled with the electrodes 1510. The circuitry 1550 can be further configured to multiplex between the electrodes 1510 to select specific electrodes (e.g., electrode pairs or pluralities of electrodes) for performing electromagnetic interrogation measurements. The circuitry 1550 can also be configured to provide signals to the electrodes 1510 for performing the electromagnetic interrogation measurements. In some implementations, the circuitry 1550 can be further configured to process the electromagnetic interrogation measurement data to assess condition of plant material being analyzed. In some implementations, the electromagnetic interrogation measurement data can be sent to a computer 1540, and the computer 1540 can then log and analyze the data to assess the condition of the associated plant material. In some implementations, the circuitry 1550 can be further configured to perform impedance tomography using feature vectors estimated from electromagnetic interrogation measurements.

By way of example, the circuitry 1550 can implement a data processing unit that is configured to store electromagnetic interrogation measurement data, such as time-domain sweeps of electrical oscillators and associated responses received through the electrodes 1510. In some implementations, the data processing unit can be configured to perform computations, such as demodulation of signals, in order to store estimated electrical impedance values.

In some implementations, an implemented data processing unit may not store measurement data. That is, the data processing unit may be operationally coupled to another computing element, e.g. the computer 1540, and/or may transmit the data wirelessly, e.g., using a Bluetooth, WiFi, or cellular connection, to another data processing unit. That is, condition assessment of the plant material may be performed on the data processing unit, or with associated software running on processing equipment that is/is not physically attached to the electrodes 1510, or to a data processing unit implemented by the computer 1540 or by the circuitry 1550.

In some arrangements, a data processing unit of the system 1500 can provide visual and/or audible feedback to a user. Such feedback can indicate when/how measurements are performed and may, or may not indicate when measurements are completed. Some data about estimated condition values determined from electromagnetic interrogation measurements can be displayed to a user, e.g., single values of impedance or estimated tomography images produced by the data processing unit. In some implementations, a data processing unit can be configured to prompt a user to enter location/plant information for data collection, or the data processing unit may record this information autonomously.

FIG. 16 is a schematic diagram illustrating a circuit 1600 that can be used for performing measurements of electrical/electromagnetic properties of growing plant material 1605. While specific circuit elements are illustrated in FIG. 16, it will appreciated that these are given by way of example. In some implementations, other circuit elements and/or other circuit arrangements can be used.

In FIG. 16, the circuit 1600 includes a digital to analog converter (DAC) 1602, a reconstruction filter 1604, a high-pass filter 1606, and a large area electrode 1608 that is in contact with, or electrically coupled with a plant material being analyzed. The circuit 1600 of FIG. 16 also includes a current sense resistor 1610, a guarded probe 1612, a voltage follower 1616, an amplifier 1618 and an analog-to-digital converter (ADC) 1620, an amplifier 1622 and an ADC 1624.

In an example implementation, the circuit of FIG. 16 can be used for performing electromagnetic interrogation measurements, and can be referred to as electromagnetic interrogation circuitry. In this example, a digital signal, e.g., a square wave, is received by the DAC 1602. The DAC 1602 can convert the received digital signal to an analog square wave signal. The reconstruction filter 1604 and the high-pass filter 1606, which can collectively be referred to as signal generation circuitry, can then convert the analog square wave signal to a zero-referenced sine wave, where the zero reference is provided by the large area electrode 1608, which also provides an electrical ground reference for the circuit 1600.

Electromagnetic interrogation measurements can be performed using measurement circuitry of the circuit 1600. For instance, a current associated with an electromagnetic interrogation measurement performed using the circuit 1600 can be determined using the current sense resistor 1610, the amplifier 1618 and the ADC 1620. A voltage associated with an electromagnetic interrogation measurement performed using the circuit 1600 (e.g., a voltage on a center probe 1613 of the guarded probe 1612) can be determined using the amplifier 1622 and the ADC 1624. In this example, a guard ring 1615 of the guarded probe 1612 can be driven by the voltage follower 1616 based on the voltage present on the center probe 1613 of the guarded probe 1612. An associated impedance can then be determined (e.g., calculated) from the current and voltage associated with the electromagnetic interrogation measurement being performed are determined.

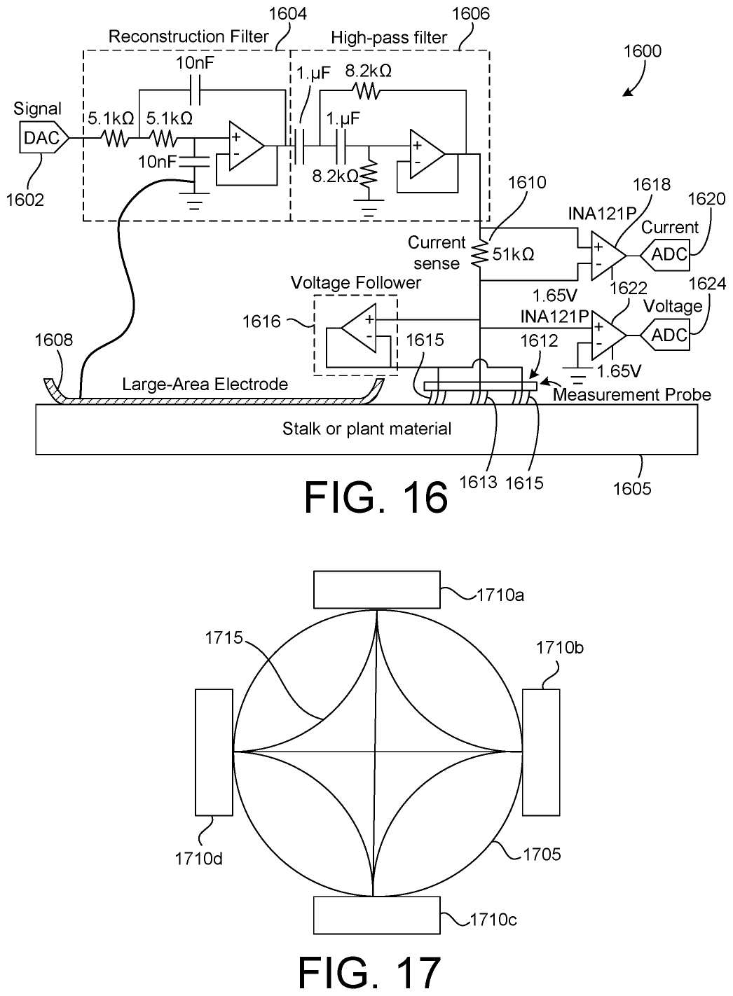

FIG. 17 is a diagram schematically illustrating a process for performing impedance tomography for condition assessment of growing plant material, e.g., a maize stalk 1705. As shown in FIG. 17, a plurality of electrodes can be circumferentially arranged around growing plant material that is being assessed. In this example, four electrodes are shown. In some implementations, additional electrodes can be included, and spacing between the electrodes can vary. That is, electrode spacing can be regular (equidistant from one electrode to the next), or can be irregular, (varying distances between adjacent electrodes).

As shown in FIG. 17, the plurality of electrodes in this example can include a first electrode 1710a, a second electrode 1710b, a third electrode 1710c and a further electrode 1710d. In FIG. 17, lines 1715 illustrate the various paths (bi-directional paths) between all four electrodes. In this example, there are 12 possible interrogation paths, three from each of the four electrodes. In some implementations, electromagnetic interrogation measurements can be taken along all 12 interrogation paths to create a map of measurements along those interrogation paths. Those measurements can then be used as inputs to an inversion routine to solve for a mathematical mesh that produces a map of the inside of the stalk (e.g., an impedance map). In other words, the relative impedances between electrodes are used to recreate an internal image of the corn stalk by solving for a mathematical mesh based on currents and voltages of respective electromagnetic interrogation measurements along each of the measured interrogation paths.

For instance, collected measurement data can be processed to evaluate the condition of growing plant material, as well as to identify plant features of interest. That collected measurement data can be used for impedance tomography, as described herein. In some implementations, impedance tomography can be performed using multiple values of the impedance that are generated through the measurement system (e.g., along available interrogation paths). The measurement system can use a signal source and measurement circuitry to estimate voltage and current through the materials that are being interrogated. While an electrode ring can be used in particular arrangements, impedance tomography can be computed on arbitrary geometries. That is, different meshing elements may be used to estimate a condition within analyzed plant material. In some implementations, electrodes used for taking electromagnetic interrogation measurements described herein can be guarded such as using the approach discussed above with respect to FIG. 16.

In a general aspect, a method can include measuring at least one electrical property of a growing plant material using a plurality of electrodes to electromagnetically interrogate the growing plant material. The method can further include, based on the measured at least one electrical property, assessing at least one physical condition of the growing plant material.

Implementations can include one or more of the following features. For example, the at least one electrical property can include an electrical impedance measurement of a portion of the growing plant material. The at least one electrical property can include measurements of electrical resistance of a portion of the growing plant material over a range of frequencies. The at least one electrical property can include measurements of capacitance of a portion of the growing plant material over a range of frequencies.

The at least one physical condition of the growing plant material can include an amount of hydration in the growing plant material. The at least one physical condition of the growing plant material can include a presence or an absence of pest damage. The growing plant material can be a maize stalk, and the pest damage can be a result of a European corn borer.

Measuring the at least one electrical property of a growing plant material can include measuring a plurality of electrical impedances of respective portions of the growing plant material. The method can include performing an impedance tomography process using the plurality of electrical impedance measurements to generate an impedance map corresponding with an internal structure of the growing plant material.

In another general aspect, a system can include an electromagnetic interrogation device that includes a plurality of electrodes configured to measure electrical properties of a growing plant material. The system can also include data processing circuitry configured to, based on the measured electrical properties, assess at least one physical condition of the growing plant material.

Implementations can include one or more of the following features. For example, the system can include electromagnetic interrogation circuitry. The electromagnetic interrogation circuitry can include signal generation circuitry configured to provide a stimulus signal to a first electrode of the plurality of electrodes. The electromagnetic interrogation circuitry can include measurement circuity configured to, receive the stimulus signal at a second electrode of the plurality of electrodes via the growing plant material, determine a current of the stimulus signal through the plant material, and determine a voltage of the stimulus signal at the first electrode. The signal generation circuitry can be configured to generate the stimulus signal across a range of frequencies. The signal generation circuitry can be configured to generate the stimulus signal at one or more fixed frequencies.

The system can include a distance encoder configured to determine a position of the electromagnetic interrogation device on the growing plant material.

The data processing circuitry can be configured to perform an impedance tomography process using the measured electrical properties to generate a map corresponding with an internal structure of the growing plant material.

The electromagnetic interrogation device can be configured to place the plurality of electrodes in physical proximity of, but spaced from the growing plant material. The electromagnetic interrogation device can be configured to place the plurality of electrodes in physical contact with the growing plant material.

The electrical properties can include resistance and capacitance. The electromagnetic interrogation device can be configured to measure the electrical properties of the growing plant material over a range of frequencies. The electromagnetic interrogation device can be configured to measure the electrical properties of the growing plant material using a plurality of combinations of the plurality of electrodes.

In the foregoing disclosure, it will be understood that when an element, such as a layer, a region, or a substrate, is referred to as being on, connected to, electrically connected to, coupled to, or electrically coupled to another element, it may be directly on, connected or coupled to the other element, or one or more intervening elements may be present. In contrast, when an element is referred to as being directly on, directly connected to or directly coupled to another element or layer, there are no intervening elements or layers present. Although the terms directly on, directly connected to, or directly coupled to may not be used throughout the detailed description, elements that are shown as being directly on, directly connected or directly coupled can be referred to as such. The claims of the application may be amended to recite exemplary relationships described in the specification or shown in the figures.

As used in this specification, a singular form may, unless definitely indicating a particular case in terms of the context, include a plural form. Spatially relative terms (e.g., over, above, upper, under, beneath, below, lower, and so forth) are intended to encompass different orientations of the device in use or operation in addition to the orientation depicted in the figures. In some implementations, the relative terms above and below can, respectively, include vertically above and vertically below. In some implementations, the term adjacent can include laterally adjacent to or horizontally adjacent to.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

What is claimed is:

1. A method comprising:
measuring at least one electrical property of a growing plant material using a plurality of electrodes to electromagnetically interrogate the growing plant material; and
based on the measured at least one electrical property, assessing at least one physical condition of the growing plant material.

2. The method of claim 1, wherein the at least one electrical property includes an electrical impedance measurement of a portion of the growing plant material.

3. The method of claim 1, wherein the at least one electrical property includes measurements of electrical resistance of a portion of the growing plant material over a range of frequencies.

4. The method of claim 1, wherein the at least one electrical property includes measurements of capacitance of a portion of the growing plant material over a range of frequencies.

5. The method of claim 1, wherein the at least one physical condition of the growing plant material include an amount of hydration in the growing plant material.

6. The method of claim 1, wherein the at least one physical condition of the growing plant material includes a presence or an absence of pest damage.

7. The method of claim 6, wherein:
the growing plant material is a maize stalk; and
the pest damage is a result of a European corn borer.

8. The method of claim 1, wherein measuring the at least one electrical property of a growing plant material includes measuring a plurality of electrical impedances of respective portions of the growing plant material, the method further comprising:
performing an impedance tomography process using the plurality of electrical impedance measurements to generate an impedance map corresponding with an internal structure of the growing plant material.

9. A system comprising:
an electromagnetic interrogation device that includes a plurality of electrodes configured to measure electrical properties of a growing plant material; and
data processing circuitry configured to, based on the measured electrical properties, assess at least one physical condition of the growing plant material.

10. The system of claim 9, further comprising measurement circuitry including:
signal generation circuitry configured to provide a stimulus signal to a first electrode of the plurality of electrodes; and
measurement circuity configured to:
receive the stimulus signal at a second electrode of the plurality of electrodes via the growing plant material;
determine a current of the stimulus signal through the growing plant material; and
determine a voltage of the stimulus signal at the first electrode.

11. The system of claim 10, wherein the signal generation circuitry is configured to generate the stimulus signal across a range of frequencies.

12. The system of claim 10, wherein the signal generation circuitry is configured to generate the stimulus signal at one or more fixed frequencies.

13. The system of claim 9, further comprising a distance encoder configured to determine a position of the electromagnetic interrogation device on the growing plant material.

14. The system of claim 9, wherein the data processing circuitry is further configured to perform an impedance tomography process using the measured electrical properties to generate a map corresponding with an internal structure of the growing plant material.

15. The system of claim 9, wherein the electromagnetic interrogation device is configured to place the plurality of electrodes in physical proximity of, but spaced from the growing plant material.

16. The system of claim 9, wherein the electromagnetic interrogation device is configured to place the plurality of electrodes in physical contact with the growing plant material.

17. The system of claim 9, wherein the electrical properties include resistance and capacitance.

18. The system of claim 9, wherein the electromagnetic interrogation device is configured to measure the electrical properties of the growing plant material over a range of frequencies.

19. The system of claim 9, wherein the electromagnetic interrogation device is configured to measure the electrical properties of the growing plant material using a plurality of combinations of the plurality of electrodes.
